(19) United States
(12) Patent Application Publication
(10) Pub. No.: US 2017/0171945 A1
(43) Pub. Date: Jun. 15, 2017

(54) Voice Controlled Medical Device and Method of Hands Off Operation

(71) Applicant: LuceLine Unlimited, Inc., Wayzata, MN (US)

(72) Inventor: Stephen D. Paulson, Orono, MN (US)

(73) Assignee: LuceLine Unlimited, Inc., Wayzata, MN (US)

(21) Appl. No.: 15/215,438

(22) Filed: Jul. 20, 2016

Related U.S. Application Data
(60) Provisional application No. 62/179,900, filed on May 21, 2015.

(51) Int. Cl. H05B 37/02; G05B 19/05; B25J 13/00; A61B 90/00

(52) U.S. Cl. CPC H05B 37/0236; A61B 90/36; G05B 19/05; B25J 13/003; A61B 2017/00203

(57) ABSTRACT

The present invention describes a voice control system to hands-off control lighting in a medical facility comprising an audio transducer in electrical communication with a control unit, the control unit configured with voice recognition software and programmable logic circuitry to generate executable commands, a lighting device configured to receive, decode, and execute commands received from the control unit, the lighting device in mechanical communication with a support structure and attached thereto by an articulated arm, the articulated arm configured to receive, decode, and execute commands received from the control unit.

### CROSS-REFERENCE TO RELATED APPLICATIONS

This application claims the benefit of U.S. Provisional Patent Application No. 62/179,900 filed May 21, 2015, which hereby is incorporated herein in its entirety by reference thereto.

### FIELD OF THE INVENTION

The present invention is directed generally to medical devices and more specifically to a method to hands-off control lighting devices used during medical procedures via voice commands.

### BACKGROUND

Voice control systems are being introduced into every aspect of human endeavor including the control of machines, computers, cell phones, navigation systems (both for motor vehicles and commercial aircraft) amongst others and would be very useful in medical procedures where adherence to sterility may require hands free operation in such places as the operating and emergency rooms.

Examples of the diversity of such prior art can be found in the following:
U.S. Pat. No. 8,996,383 issued Mar. 31, 2015 entitled “Motor-Vehicle Voice-Control System and Microphone-Selecting Method Therefor.”
U.S. Pat. No. 8,793,139 issued Jul. 29, 2014 entitled “Voice Activated Cockpit Management System for Flight Procedures and Control of Aircraft Systems and Flight Management Systems of Single and Multi-Engine Aircraft.”
U.S. Pat. No. 7,921,017 issued Apr. 5, 2011 entitled “Systems and Methods for Voice Control of a Medical Device” which describes the voice control of devices used in phacoemulsification (i.e. cataract) surgery including the variable speed pump for providing a vacuum source and a pulsed ultrasonic power source for providing control outputs to a pump speed controller and an ultrasonic power level controller.
US Patent Application Publication No. 2015/0120298 published Apr. 30, 2015 entitled “Voice Control System for an Implant” which describes voice control of such items as insulin pumps and pacemakers amongst other various implantable devices.
US Patent Application Publication No. 2014/0371555 published Dec. 18, 2014 entitled “System and Method for Voice Control of Medical Devices” which describes voice control of optical instruments external to the human body to noninvasively measure parameters such as blood oxygen level, blood pressure, and heart rate amongst other physiologic parameters.

### SUMMARY OF THE INVENTION

The present invention is directed generally to medical devices and more specifically to a method to hands-off control lighting devices used during medical procedures via voice commands.

One embodiment of the present invention describes a voice control system to hands-off control lighting in a medical facility comprising an audio transducer in electrical communication with a control unit, the control unit configured with voice recognition software and programmable logic circuitry to generate executable commands, a lighting device configured to receive, decode, and execute commands received from the control unit, the lighting device in mechanical communication with a support structure and attached thereto by an articulated arm, the articulated arm configured to receive, decode, and execute commands received from the control unit, and the articulated arm motor driven and capable of rotation about 3 orthogonal axes and can extend inward or outward from said support structure in response to appropriate commands received from the control unit.

Another embodiment of the present invention describes a voice control system to hands-off control lighting in a medical facility comprising an audio transducer in electrical communication with a control unit, the control unit configured with voice recognition software and programmable logic circuitry to generate executable commands, a lighting device configured to receive, decode and execute commands received from the control unit, the lighting device in mechanical communication with a support structure and attached thereto by an articulated arm, the articulated arm configured to receive, decode, and execute commands received from the control unit, the articulated arm motor driven and capable of rotation about 3 orthogonal axes and can extend inward or outward from said support structure in response to appropriate commands received from the control unit, a video device configured to receive, decode, and execute audio commands received from the control unit, and the articulated arm motor driven and capable of rotation about 3 orthogonal axes and can extend inward or outward from said support structure in response to appropriate voice commands.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A shows one embodiment of the present invention depicting a physician initially observing a patient in a medical exam room configured with a voice activated lighting device initially shown in the fully retracted position.

FIG. 1B shows the same medical exam room after the physician has given a verbal command for the lighting device to “zoom in” and the lighting device has transitioned to its fully extended position.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The present invention is directed generally to medical devices and more specifically to a method to control lighting devices used during medical procedures via voice commands.

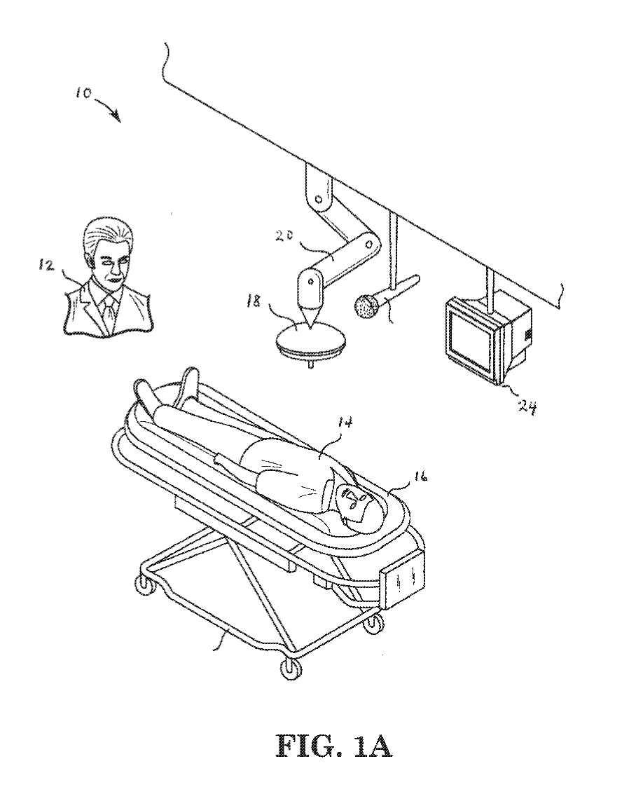

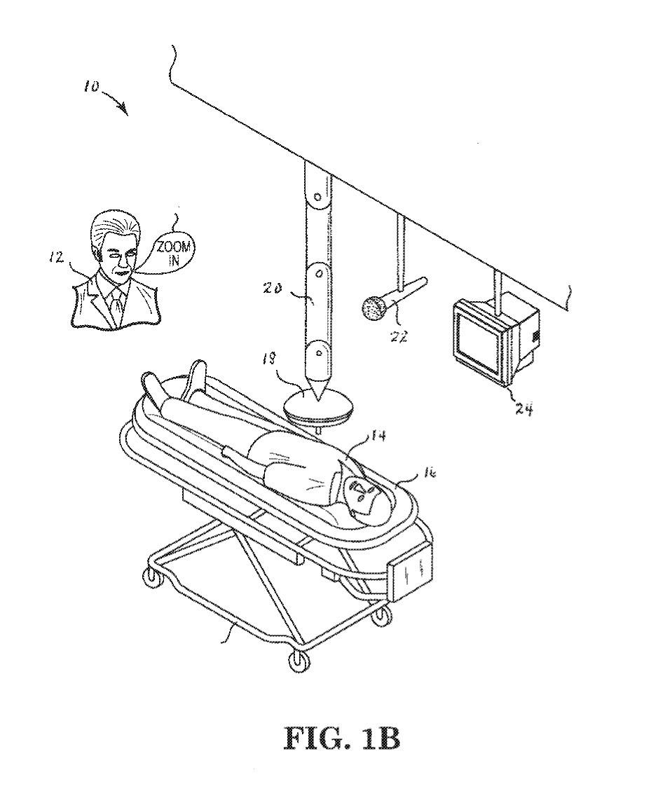

One embodiment of the present invention is depicted in FIG. 1A.

FIG. 1A shows a medical exam room 10 with a physician 12 viewing a patient 14 who is lying on a typical examination table 16. The exam room 10 is shown with an overhead lighting device 18 which may be attached to a motor driven articulated arm 20, which in turn may be attached to the ceiling of the exam room 10 or any appropriate overhead support member.

The exam room may also include a microphone 22, a controller unit (not shown in FIG. 1A), and a video display device 24 all of which may also be located proximate to the articulated arm 20 and may be attached to the same overhead support member. The controller unit may be in electrical communication with the microphone 22, the video display device 24, and the motor(s) which actuate movement of the articulated arm 20. In one embodiment of the present invention, the controller unit may be configured with voice recognition software and can be programmed to interpret incoming audio commands from the microphone 22 as generated by the physician 12. In addition, the controller unit may also incorporate programmable logic circuitry (PLC) which may convert the output of the voice recognition software into executable commands to both the motor(s) which actuate movement of the articulated arm 20 and the power supply in the overhead lighting device 18 which controls the light level output.

Returning to FIG. 1A—this may depict the case where the physician has just entered the exam room 10 and is about to begin the exam. Initially as shown, the articulated arm 20 may be in the retracted “home” position and the overhead lighting device may be off. The programmable logic circuitry (PLC) may be programmed to decode and execute a myriad of voice commands from the physician. Those commands relating to the overhead lighting device 18 may include any of the following separately or in combination: “turn lighting on, turn lighting off, increase lighting, decrease lighting, stop increasing lighting, stop decreasing lighting, go to lighting level 1, go to lighting level 2, and stop.”

In addition, the voice recognition software in concert with the programmable logic circuitry (PLC) may decode and generate executable commands simultaneously directed to the motors which drive the articulated arm 20. Those commands relating to the articulated arm 20 may include any of the following separately or in combination: “go home, shift left, shift right, shift up, shift down, shift left one unit, shift right one unit, shift up one unit, shift down one unit, zoom in, zoom out, zoom in one unit, zoom out one unit, rotate clockwise, rotate counterclockwise, rotate clockwise one unit, rotate counterclockwise one unit, rotate up, rotate down, rotate up one unit, rotate down one unit, go to setting one, go to setting two, and stop.”

FIG. 1B shows the results of the physician entering the exam room 10 and issuing the voice command “zoom in” wherein the voice recognition software has correctly deciphered the command, and the programmable logic circuitry (PLC) has delivered the appropriate drive signals to the motors which actuate movement of the articulated arm 20 and the result as shown in FIG. 1B is the articulated arm 20 in its' fully extended position.

In an alternative embodiment of the present invention, the voice recognition software and the programmable logic circuitry (PLC) may be included along with the microphone into one integrated device which performs all of the functions described above. Even more, in a preferred embodiment of the present invention, the hands-off voice activated system would also include the video monitor 24 depicted in FIGS. 1A and 1B. In the event the hospital's archived patient records could be accessed through voice commands via the voice recognition software and the programmable logic circuitry (PLC) working in concert with the hospital's central data base, the attending physician could, for example, review the patient's most recent lab results, request additional staff support, or in an extreme case issue a hands-off verbal code blue alert.

The present invention should not be considered limited to the particular examples described above, but rather should be understood to cover all aspects of the invention as fairly set out in the attached claims. Various modifications to the shape and form factors described above, equivalent processes to which the present invention may be applicable will be readily apparent to those of skill in the art to which the present invention is directed upon review of the present specification. For example, the present invention anticipates that additional features of the lighting device(s) may be modified by voice control including but not limited to utilizing multiple lighting devices with unique and separable voice commands connected to separate articulated arms such that multiple locations of the patient can be simultaneously illuminated or incorporating imaging/magnifying optics into the lighting device under voice control which can vary the size and orientation of the illuminated patient area. The following claims are intended to cover such modifications and devices.

I claim:

1. A voice control system to hands-off control lighting in a medical facility comprising:
an audio transducer in electrical communication with a control unit;
said control unit configured with voice recognition software and programmable logic circuitry to generate executable commands;
a lighting device configured to receive, decode, and execute commands received from the control unit;
said lighting device in mechanical communication with a support structure and attached thereto by an articulated arm;
said articulated arm configured to receive, decode, and execute commands received from the control unit; and
said articulated arm motor driven and capable of rotation about 3 orthogonal axes and can extend inward or outward from said support structure in response to appropriate commands received from the control unit.

2. The system of claim 1 wherein the medical facility may be an operating room, an emergency room, a patient screening area, or any imaging dedicated area.

3. The system of claim 1 wherein the means to receive an audio command may include a microphone or an equivalent audio transducer.

4. The system of claim 1 wherein the audio commands received and decoded by the control unit directed to the lighting device may include any of the following separately or in combination: turn lighting on, turn lighting off, increase lighting, decrease lighting, stop increasing lighting, stop decreasing lighting, go to lighting level 1, go to lighting level 2, and stop.

5. The system of claim 1 wherein the audio commands received and decoded by the control unit directed to the articulated arm may include any of the following separately or in combination: go home, shift left, shift right, shift up, shift down, shift left one unit, shift right one unit, shift up one unit, shift down one unit, zoom in, zoom out, zoom in one unit, zoom out one unit, rotate clockwise, rotate counterclockwise, rotate clockwise one unit, rotate counterclockwise one unit, rotate up, rotate down, rotate up one unit, rotate down one unit, go to setting one, go to setting two, and stop.

6. The system of claim 1 wherein the means to decode said audio command may include DXM 512 or equivalent software algorithms.

7. A voice control system to hands-off control lighting in a medical facility comprising;
an audio transducer in electrical communication with a control unit;
said control unit configured with voice recognition software and programmable logic circuitry to generate executable commands;
a lighting device configured to receive, decode, and execute commands received from the control unit;
said lighting device in mechanical communication with a support structure and attached thereto by an articulated arm;
said articulated arm configured to receive, decode, and execute commands received from the control unit; and
said articulated arm motor driven and capable of rotation about 3 orthogonal axes and can extend inward or outward from said support structure in response to appropriate commands received from the control unit;
a video device configured to receive, decode, and execute audio commands received from the control unit; and
said articulated arm motor driven and capable of rotation about 3 orthogonal axes and can extend inward or outward from said support structure in response to appropriate voice commands.
